(19) United States
(12) Patent Application Publication
(10) Pub. No.: US 2023/0309888 A1
(43) Pub. Date: Oct. 5, 2023

(54) APPARATUS AND METHOD FOR HYBRID BIOSENSORS

(71) Applicant: Roger Young, Portland, OR (US)

(72) Inventor: Roger Young, Portland, OR (US)

(21) Appl. No.: 18/126,741

(22) Filed: Mar. 27, 2023

Related U.S. Application Data
(60) Provisional application No. 63/325,065, filed on Mar. 29, 2022.

(51) Int. Cl. A61B 5/25; A61N 1/04

(52) U.S. Cl. CPC A61B 5/25; A61N 1/04

(57) ABSTRACT

A system and method for securing a hybrid sensor to a patient's skin is provided. One embodiment has a dry sensor component that is configured to be in electrical contact with the patient's skin after the dry sensor component has been secured to the patient's skin. A wet sensor lead component is configured to be secured to the dry sensor component after the dry sensor component has been secured to the patient's skin. The wet sensor lead component, when communicatively coupled to the dry sensor component and to the amplifier, communicates voltage information sensed by the dry sensor component to an amplifier.

### PRIORITY CLAIM

This application claims priority to copending U.S. Application, Ser. No. 63/325,065, filed on Mar. 29, 2022, entitled Apparatus and Method For Hybrid Biosensors, which is hereby incorporated by reference in its entirety for all purposes.

### BACKGROUND OF THE INVENTION

Biosensors may be used to sense various conditions of a patient. Electrodes may be used to stimulate organs of the patient. Both biosensors and electrodes require making a low impedance electrical connection using a physical connection to the patient or otherwise being in a close proximity to allow recording bio signals.

Passive biosensors receive signals produced by the body. Voltages are then sent through a lead connected to the biosensor to an amplifier. The voltages may then be processed by an analytic device, a display, and/or a data storage device. The Voltage signals are processed for clinical interpretation. Example passive sensors can be used to assess the brain (electroencephalogram, EEG), heart (electrocardiography, ECG) and muscle (electromyography, EMG).

Some types of example legacy sensors are classified as wet sensors. FIG. 1 illustrates an example wet sensor 10. They are commonly constructed using a conductive gel 12 that adheres to the patient's skin, a silver tape or pellet 14 to convert ionic currents and voltage to electrical currents and voltages (and vice versa). Wet sensors 10 may also include a mechanical backing 16 to provide structural support, and an optional snap or other mechanical connector 18 mechanism to provide electrical continuity between the silver tape or pellet 14 and a wire lead 20. The wire lead 20 may be permanently affixed to the sensor 10 (i.e., solder or other permanent connection) or it may be detachable (i.e., snap, clip or other mechanical connector). The backing 16 is commonly constructed with an overlap and adhesive 22 to contribute mechanical stability between the gel 12 and the skin of the patient. An optional sponge 24 may be used to restrict the gel 12 to a desired location.

Sensors constructed with gel 12 are called wet sensors because contact with the skin is made with an aqueous electrolyte gel solution, such as potassium chloride. The chloride ion is required to participate in an electrochemical reaction with the silver that creates the electrical voltage in the silver tape or pellet 14. Stability between the wet sensor 10 and the skin is accomplished by adherence properties of the gel 12 or the backing 16, or both. To match specific clinical demands, gels 12 of different viscosities and adherence properties are commercially available. Wet sensors 10 are most often considered single-use, or disposable devices, because they change recording properties if removed and reapplied. Wet sensors 10 are relatively inexpensive because the manufacturing steps do not require precision, allowing mass production to reduce cost per unit.

Wet sensors 10 present three clinical problems: 1) the gel 12 requires abrasive preparation of the patient's skin, which is uncomfortable and can be inadequately performed; 2) during prolonged recording, the gel 12 may dry and adversely affect recording; 3) gel sensors 10 are prone to detach, or partially detach, which adversely affects recording by creating electrical noise.

Dry sensors may be used to avoid gel-associated problems. Here, a “thin film dry sensor”, or simply a “dry sensor”, detects bioelectrical signals without gel, moisture, or any liquids. The terms “flexible”, “stretchable”, and “wearable” have also been used to refer to dry sensors. Dry sensors do not function using the same mechanisms as wet sensors (capacitive coupling versus Ag—AgCl chemical reaction used by wet sensors). However, these technical differences do not present functional differences in most applications.

Historically, some sensors may be constructed of a thick solid metal plate (with or without gel) that is inflexible and is pressed against the skin to make electrical contact, which has also been called a dry sensor. In this case, salt and moisture that originated from the patient's skin may provide electrical continuity between the metal and skin. In general, these sensors do not have the sensitivity and stability required for diagnostics or therapeutics. In this disclosure, include such inflexible thick metal sensors are not included as dry sensors.

To date, dry sensors have not penetrated the medical diagnostics market. So, it is not possible to define a “legacy” dry sensor. However, several offerings have been proposed. One example of a dry sensor employs a gold film. As a metal, gold is conductive and malleable. As a thin film, slightly modified gold adheres to the skin and functions as a dry sensor.

Another example dry sensor 30 employs graphene 32, which is two-dimensional carbon one carbon atom thick) and conducts electricity well. Similar to graphene is a slightly thicker carbon film which also conducts electricity well. One published example of a graphene-based or thin carbon film dry sensor 30 is illustrated in FIG. 2. A backing 34 may be used, which mechanically reinforces the contact between the thin film electrode 32 and the wire lead 36. Currently there are no descriptions of devices that make electrical continuity between the dry sensor 30 and the wire lead without providing permanent mechanical support and permanent electrical continuity between the dry sensor and the wire. As used herein, the term graphene 32 refers to all films composed primarily of graphene, such as chemically modified graphene, and/or graphene or carbon composites. Chemically modified graphene is capable of sensing specific molecules.

Other examples of dry sensors include films made of metal nanoparticles or nanowires. Conductive films such as polypyrole, polypyrene, or polydimethylsiloxane are thicker and less fragile than graphene. Some conductive films exhibit excellent flexibility properties and will be included in our definition of a “thin film” dry sensor. The additional thickness confers more mechanical stability while maintaining flexibility. All thin film dry sensors are mechanically fragile. Discussions of dry sensors herein refers to all types of thin film dry sensors.

Advantages of dry sensors include: 1) are functional for hours, days, or weeks without degradation of recording characteristics; 2) abrasive skin prep is not required; 3) minimal damage or irritation to skin even with prolonged use; 3) may be easily applied, or self-applied, with minimal training using an adhesive tape 40 or the like; 4) produce stable recordings and are resistant to motion artifact; 5) when worn, patients do not feel the sensors, and they are not visually obtrusive.

Disadvantages of dry sensors include: 1) a mechanical interface 38 between a two-dimensional or other thin film and a three-dimensional lead 36 causes the point of connection to be mechanically fragile; 2) difficulty upscaling methods to connect leads 36, which increases cost of manufacturing; 3) damaged beyond functionality if removed since dry sensors cannot be reused.

In summary, dry sensors exhibit significant advantages of functionality, safety, and end-user acceptability over wet sensors. However, overcoming manufacturing costs and single-use limitations of dry sensors are significant barriers to developing a compelling market advantage over wet sensors.

Active sensors, interchangeably referred to as electrodes, provide electrical connectivity between a voltage and/or current source and the electrode which is secured to the patient. In some situations, current and/or voltage pass through the electrode into the patient. Electrodes may be used to test muscle function, activate muscles, or inhibit nerve functioning.

Accordingly, in the arts of biosensors and electrodes, there is a need in the arts for improved methods, apparatus, and systems for biosensors and electrodes.

### SUMMARY OF THE INVENTION

A system and method for securing a hybrid sensor to a patient's skin is provided. One embodiment has a dry sensor component that is configured to be in electrical contact with the patient's skin after the dry sensor component has been secured to the patient's skin. A wet sensor lead component is configured to be secured to the dry sensor component after the dry sensor component has been secured on to or close to the patient's skin. The wet sensor lead component, when communicatively coupled to the dry sensor component and to the amplifier, communicates voltage information sensed by the dry sensor component to an amplifier.

### BRIEF DESCRIPTION OF THE DRAWINGS

The components in the drawings are not necessarily to scale relative to each other. Like reference numerals designate corresponding parts throughout the several views.

FIG. 1 is a diagram of a legacy wet sensor.

FIG. 2 is a diagram of a legacy dry sensor.

FIG. 3 is a diagram of an example hybrid biosensor.

FIG. 4 is a cross sectional side view diagram of an example embodiment of the hybrid biosensor.

FIGS. 5A-5D are diagrams of various embodiments of the dry sensor component.

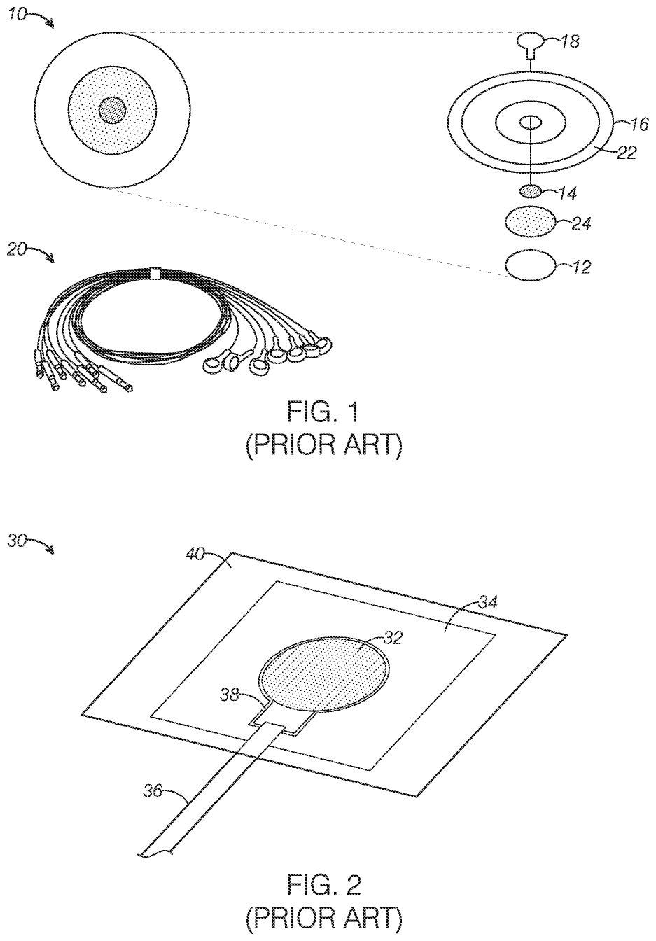

### DETAILED DESCRIPTION

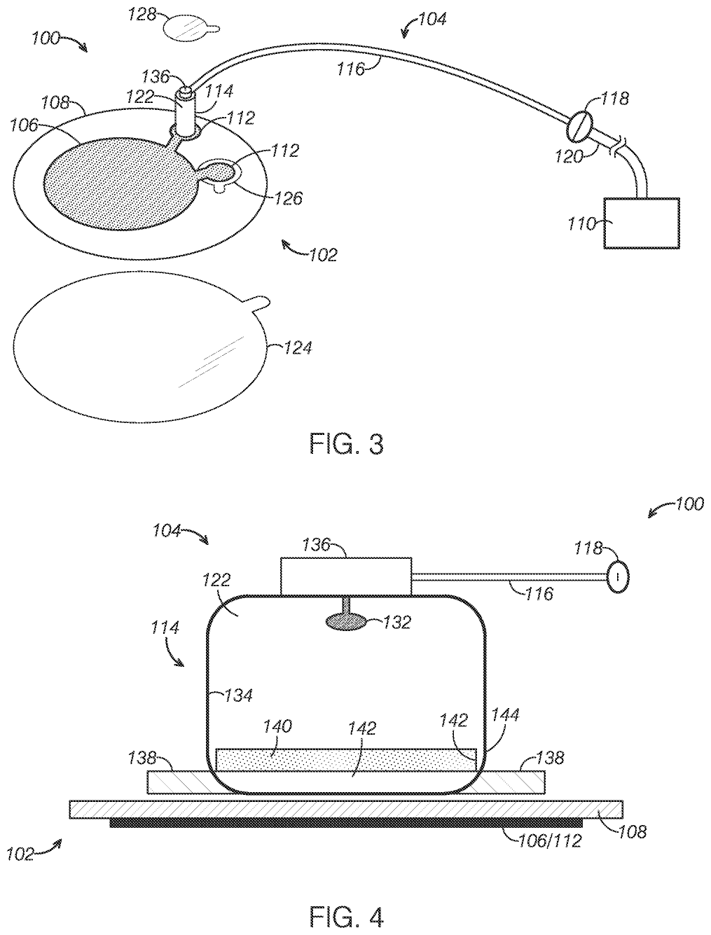

FIG. 3 is a diagram of an example hybrid biosensor 100. Embodiments of the hybrid biosensor 100 comprise a dry sensor component 102 and a wet sensor lead component 104. Embodiments of the hybrid biosensor 100 provides a system and method for providing the advantages of dry sensors, while using properties of wet sensors to overcome the disadvantages of dry sensors. Embodiments of the hybrid biosensor 100 are fully functional without signal degradation over time; are safe and skin-friendly; are capable of precision shaping; are easy to apply; are mechanically robust during use; are capable of recording repetitively; and are manufactured using systems that are up-scalable and cost-competitive with legacy wet sensors.

An electrically conductive element 106 on a lower side of the dry sensor component 102 is secured to and is in physical contact with a patient's skin when the dry sensor component 102 has been secured to the patient's skin. In a preferred embodiment, the electrically conductive element 106 is constructed as a thin film or thin material layer with minimal or no mechanical support. The material of the electrically conductive element 106 may be made of any suitable conductive material. Preferably, the electrically conductive element 106 may be flexible, or partially flexible, so that when the dry sensor component 102 is secured to the patient's skin, the deformable electrically conductive element 106 conforms to the surface profile of the patient's skin so as to maximize physical and electrical contact between the patient's skin and the lower surface of the electrically conductive element 106.

An example embodiment uses graphene for the electrically conductive element 106, which is two-dimensional carbon and conducts electricity well. Other non-limiting examples of the electrically conductive element 106 include films made of carbon, metal nanoparticles or nanowires. Conductive films such as polypyrole, polypyrene, or polydimethylsiloxane are thicker and less fragile than graphene may be sued as the electrically conductive element 106. In a preferred embodiment, the electrically conductive element 106 may be a film that exhibits excellent flexibility properties. The additional thickness of some embodiments of the electrically conductive element 106 confer more mechanical stability while maintaining flexibility.

Some embodiments of the dry sensor component 102 may use an optional thick film covering 108 to enhance support and to protect the electrically conductive element 106 from inadvertent physical and/or moisture damage. The thick film covering 108 covers at least the top surface of electrically conductive element 106 and provides support and protection to the electrically conductive element 106. The thick film covering 108 may or may not be conductive depending upon the embodiment. If the thick film covering 108 is conductive, the wet sensor cup can be secured directly to the top surface of the thick film covering 108. If the thick film covering 108 is not electrically conductive (is an electric insulator), a portion of the thick film covering is removeable by the practitioner so as to expose the electrically conductive element 106 for coupling to the wet sensor cup 114. Preferably, the thick film covering 108 is a flexible fabric material. Optionally, the thick film covering 108 is pre-sterilized (along with the electrically conductive element 106) so that the dry sensor component 102 may be packaged in a sterilized package prior to use.

In some embodiments, an adhesive around at least the outside perimeter of the thick film covering 108 may be used to secure the dry sensor component 102 to a patient. In some embodiments, one or more apertures in the thick film covering 108 expose the top surface of the electrically conductive element 106 (or one of the dry conductor leads 112) to facilitate electrical coupling of the wet sensor lead component 104 to the dry sensor component 102. Alternatively, or additionally, the thick film covering 108 may be perforated and/or may be liquid permeable to allow passing of the electrically conductive wet gel 122 through the thick film covering 108 after the wet sensor lead component 104 has been secured to the dry sensor component 102.

After the dry sensor component 102 has been secured to the patient's skin so that the electrically conductive element 106 is in electrical contact with the patient's skin, the wet sensor lead component 104 is then secured to a selected portion of the electrically conductive element 106 of the dry sensor component 102. Accordingly, the wet sensor lead component 104 is not required to contact the patient's skin. Further, since the wet sensor lead component 104 is electrically coupled to the dry sensor component 102 after the dry sensor component 102 has been secured to the patient's skin, the wet sensor lead component 104 can be electrically coupled to the dry sensor component 102 at any selected time of interest during an examination and/or can be electrically coupled to the dry sensor component 102 at a different location than where the dry sensor component 102 was secured to the patient's skin.

The wet sensor lead component 104 creates electrical continuity between the dry sensor component 102 and an amplifier 110, or other electronic device that includes an internal amplifier 110, that senses at least voltage signals generated and communicated by the hybrid biosensor 100. That is, when the wet sensor lead component 104 is coupled to the dry sensor component 102 and to the amplifier 110, voltage of the patient's skin is sensed at the electrically conductive element 106 of the dry sensor component 102. The sensed voltage (interchangeably referred to herein as voltage information) is communicated from the hybrid biosensor 100 to the amplifier 110. Together, the dry sensor component 102 and the wet sensor lead component 104 function as a hybrid EEG/ECG/EMG sensor/electrode, referred to herein as a hybrid biosensor 100.

The disclosed systems and methods for a hybrid biosensor 100 will become better understood through review of the following detailed description in conjunction with the figures. The detailed description and figures provide examples of the various inventions described herein. Those skilled in the art will understand that the disclosed examples may be varied, modified, and altered without departing from the scope of the inventions described herein. Many variations are contemplated for different applications and design considerations, however, for the sake of brevity, each and every contemplated variation is not individually described in the following detailed description.

Throughout the following detailed description, a variety of examples for systems and methods for a hybrid biosensor 100 are provided. Related features in the examples may be identical, similar, or dissimilar in different examples. For the sake of brevity, related features will not be redundantly explained in each example. Instead, the use of related feature names will cue the reader that the feature with a related feature name may be similar to the related feature in an example explained previously. Features specific to a given example will be described in that particular example. The reader should understand that a given feature need not be the same or similar to the specific portrayal of a related feature in any given figure or example.

The following definitions apply herein, unless otherwise indicated.

There are varying definitions of the terms “sensor” and “electrode”. Commonly, a “passive sensor” describes passively recording a signal, while an “active electrode” refers to passing a current through the electrode device to elicit a function, such as a muscle contraction or release of a medication. The prefix “bio-” refers to the use of these devices on biologic systems. In the following disclosure, the term “sensor” to refers to both or either of a passive sensor and/or active electrode, while “electrode” will be used to describe specific characteristics of active electrodes.

“Substantially” means to be more-or-less conforming to the particular dimension, range, shape, concept, or other aspect modified by the term, such that a feature or component need not conform exactly. For example, a “substantially cylindrical” object means that the object resembles a cylinder, but may have one or more deviations from a true cylinder.

“Comprising,” “including,” and “having” (and conjugations thereof) are used interchangeably to mean including but not necessarily limited to, and are open-ended terms not intended to exclude additional, elements or method steps not expressly recited.

Terms such as “first”, “second”, and “third” are used to distinguish or identify various members of a group, or the like, and are not intended to denote a serial, chronological, or numerical limitation.

“Coupled” means connected, either permanently or releasably, whether directly or indirectly through intervening components. “Secured to” means directly connected without intervening components.

“Communicatively coupled” means that an electronic device exchanges information with another electronic device, either wirelessly or with a wire based connector, whether directly or indirectly through a communication network. “Controllably coupled” means that an electronic device controls operation of another electronic device.

“Electrical contact” and/or “electrically coupled” means that one element of the hybrid biosensor 100 is electrically coupled to a second element such that voltage of both elements are the same. That it, the two elements in electrical contact with each other are communicatively coupled together such that voltage signals may be transferred over the electrically coupled elements. Electrical contact includes the capacitive coupling of an electrically conductive element (or one of the dry conductor leads) of a dry sensor component 102 with the patient's skin.

An “electrode” is defined as an electrical conductor, typically made of metal or other conducting material. An electrode is used to make electrical contact with a nonmetallic element (such as a conducting wet gel) of an electrical circuit.

Returning to FIG. 3, the electrically conductive element 106 of the dry sensor component 102 is preferably made of any flexible, or semi-flexible, thin film of electrically conductive material such as, but not limited to, graphene, malleable metal, or polymer film. Alternatively, the electrically conductive element 106 may be rigid. Various methods of fabrication of the dry sensor component 102 are acceptable. Any dry sensor component 102 now known or later developed may be used in the various embodiments, and are intended to be included within the scope of this disclosure and to be protected by the accompanying claims.

In some embodiment, one or more optional dry conductor leads 112 (interchangeably referred to herein as a peninsula 112) may extend outwardly from the body portion of the electrically conductive element 106. The dry conductor leads 112 facilitate a single use coupling of the wet sensor lead component 104 to the dry sensor component 102 after the dry sensor component 102 has been secured to the patient's skin at a desired location on their body. The dry conductor leads 112 permit the covering and protection of the body portion of the electrically conductive element 106 using the thick film covering 108, surgical tape, or the like.

In an example embodiment, the outside perimeter of the thick film covering 108 optionally extends beyond the perimeter of the electrically conductive element 106 and includes a suitable adhesive on the extended outside perimeter so that the dry sensor component 102 can be secured to the patient's skin. Alternatively, or additionally, a surgical tape or the like can be used to secure the dry sensor component 102 to the patient's skin, particularly if one or more of the dry conductor leads 112 extend outwardly beyond the edges of the surgical tape. Alternatively, or additionally, and electrically conductive adhesive may be used to secure the dry sensor component 102 to the patient's skin.

The wet sensor lead component 104 may include a wet sensor cup 114 coupled to a proximal end of a wet sensor lead wire 116. An optional connector 118 is coupled to the distal end of the wet sensor lead wire 116. The connector 118 is configured to easily provide electrical coupling to a wire connector 120 that extends back to the amplifier 110 that receives and processes voltages, or changes in voltages, sensed by the hybrid biosensor 100. Various methods of fabrication of the wet sensor component 104 are acceptable. Any wet sensor component 104 now known or later developed may be used in the various embodiments, and are intended to be included within the scope of this disclosure and to be protected by the accompanying claims. Any suitable connector 118 may be used in the various embodiments.

To create electrical continuity between the dry sensor component 102 and the wet sensor lead component 104, an interior of the wet sensor cup 114 contains a viscous electrically conductive wet gel 122. The electrically conductive wet gel 122 enables a low impedance electrical connection with the electrically conductive element 106 of the dry sensor component 102.

FIG. 4 is a cross sectional side view diagram of an example embodiment of the hybrid biosensor 100. In a non-limiting example embodiment, the wet sensor cup 114 contains an Ag/AgCl (silver electrode) wire or pellet 132 (interchangeably referred to herein as an electrode 132). In other embodiments, the electrode 132 may be made of any suitable electrically conductive metal or material.

The interior portion of the wet sensor cup 114 is defined by a cup wall 134 that holds (retains) a viscous electrolyte gel 122. The electrically conductive wet gel 122 fills the wet sensor cup 114 so as to encompass the electrode 132. Accordingly, the electrode 132 and the electrically conductive wet gel 122 are electrically coupled together.

Once the dry sensor component 102 of the hybrid biosensor 100 has been secured to the patient's skin, a portion of the electrically conductive wet gel 122 exits from the bottom opening (interchangeably referred to herein as a cup aperture) of the wet sensor cup 114 and comes into contact with the electrically conductive element 106 (or one of the dry conductor leads 112). The opening of the wet sensor cup 114 may vary in size, and in some embodiments, may be very small (1 mm in diameter or less). Accordingly, the electrical coupling of the electrode 132 via the electrically conductive wet gel 122 provides electrical continuity between the silver electrode 132 and the electrically conductive element 106 (or one of the dry conductor leads 112).

Further, the electrode 132 is electrically and communicatively coupled to the wet sensor lead wire 116. Alternatively, or additionally, the wet sensor lead wire 116 may be coupled to a wire and/or electrically coupled to the electrically conducting gel 122 using another structure.

Some embodiments of the wet sensor cup 114 may include a connector 136 secured to an outside surface of the cup wall 134. The connector 136 provides structural support and provides electrical continuity between the Ag/AgCl electrode 132 and wet sensor lead wire 116. Accordingly, the rigid or semi-rigid wall 134 of the wet sensor cup 114 provides supporting structure for the connector 136. The connector 136 may be secured to the top surface of the wet sensor cup 114 using any suitable means, such as an adhesive, a clip, a screw or a bolt. In some embodiments, the connector 136 may be formed as a portion of s unibody wet sensor cup 114.

The point of electrical connectivity between the wet sensor lead component 104 and the dry sensor component 102 occurs when the electrically conductive wet gel 122 comes into physical contact with the electrically conductive element 106 (or one of the dry conductor leads 112). The patient's skin is not required to be in direct contact with the gel or salt solution 122 of the wet sensor lead component 104, and hence, the patient's skin does not require abrasive preparation as is required with legacy wet sensor technologies.

The wet sensor cup 114 does not necessarily need to be in the shape of an enclosing cup. In alternative embodiments, the wet sensor cup 114 may be any shape or structure that will stably hold and/or retain the electrically conductive wet gel 122 in direct contact with both the electrically conductive element 106 (or one of the dry conductor leads 112) of the dry sensor component 102 and the electrode 132.

An optional lip 138 on the wet sensor cup 114 may optionally contain an adhesive layer that secures the bottom of the wet sensor cup 114 to the top of the electrically conductive element 106 (or one of the dry conductor leads 112). The lip 138 around the perimeter of the bottom of the wet sensor cup 114 may be used to contribute to the mechanical stability between the wet sensor cup 114 and electrically conductive element 106 (or one of the dry conductor leads 112) of the dry sensor component 102.

In some embodiments, an optional sponge 140 may be used to retain the electrically conductive wet gel 122 within a wet sensor cup 114 prior to use. Alternately or additionally, a ridge or ridges on the bottom of the lip 138 may be used to retain the electrically conductive wet gel 122 within a wet sensor cup. In an example embodiment, an outer perimeter edge 142 of the sponge 140 substantially corresponds to an inside of a perimeter 144 of the bottom of the wet sensor cup 114. After the wet sensor cup 114 has been filled by some predefined amount of electrically conductive wet gel 122, the sponge 140 may be inserted into the bottom of the wet sensor cup 114. The sponge 140 may be frictionally retained within the bottom interior of the wet sensor cup 114 so that the electrically conductive wet gel 122 is retained in the interior of the wet sensor cup 114. Alternatively, or additionally, an adhesive may be used to secure the sponge to the wet sensor cup 114. Alternatively, the outside perimeter edge 142 of the sponge 140 may be the same or greater than an exterior of the perimeter 144 of the wet sensor cup 114, and may be secured to the bottom of the wet sensor cup 114 using a suitable adhesive, clip or the like.

When the wet sensor lead component 104 is secured to the dry sensor component 102, the viscous electrically conductive wet gel 122 is able to pass through the sponge 140 so as to come into electrical and physical contact with the electrically conductive element 106 (or one of the dry conductor leads 112).

In a preferred embodiment, the sponge 140 is a porous material that substantially resists flow of the viscous electrically conductive wet gel 122 prior to use. The sponge 140 may be a soft, flexible porous material. Any suitable porous material may be used, particularly in view of the viscosity of the electrically conductive wet gel 122. The porosity of the sponge 140 is designed so as to retain a sufficient amount of the viscous electrically conductive wet gel 122 within the interior of the wet sensor cup 114 so that the retained portion of the electrically conductive wet gel 122 remains in electrical contact with the electrode 132 during use.

In some embodiments, the wet sensor cup 114 is formed from the sponge material (is the sponge 140). In such embodiments, the sponge 140 may be a substitute for the wet sensor cup 114 as a mechanism to retain the electrically conductive wet gel 122 between the silver electrode 132 and the electrically conductive element 106 (or one of the dry conductor leads 112) of the dry sensor component 102. The sponge type wet sensor cup 114 will serve to provide structural support for components that provide electrical continuity between the electrically conductive element 106 (or one of the dry conductor leads 112), the Ag/AgCl electrode 132, and the wet sensor lead wire 116.

In some embodiments, the electrode 132 may be incorporated into the sponge 140. This embodiment will allow the size of the sponge 140 to be reduced. Alternatively, or additionally, the material of the sponge 140 may be electrically conducting in some embodiments.

The electrically conductive wet gel 122 may be any viscous, conductive EEG/ECG/EMG gel or paste, such as, but not limited to, Ten20 EEG conductive paste (Weaver) or Electro-gel for electro-caps (ECI). Alternately, saline or other salt electrolyte solution may be substituted for the viscous electrically conductive wet gel 122. Any suitable electrically conductive wet gel 122 may be used in the various embodiments. The viscosity of the electrically conductive wet gel 122 may be defined, in part, based on the characteristics of the wet sensor cup 114. For example, the viscosity of the electrically conductive wet gel 122 may be relatively high if the sponge 140 is not used to retain the electrically conductive wet gel 122. Alternatively, a higher viscosity electrically conductive wet gel 122 may be used in embodiments that do not employ the sponge 140.

In some embodiments that employ the sponge 140, a slight gap 142 between the bottom surface of the sponge 140 and the top surface of the electrically conductive element 106 (or one of the dry conductor leads 112) may initially exist prior to use. The practitioner may push downward onto the wet sensor cup 114 and/or squeeze the wet sensor cup 114 so that the semi rigid wall 134 of the wet sensor cup 114 deforms to urge a portion of the electrically conductive wet gel 122 downward through the sponge 140 and outward through the bottom opening of the wet sensor cup 114 so that the portion of the electrically conductive wet gel 122 comes into electrical contact the electrically conductive element 106 (or one of the dry conductor leads 112).

In a preferred embodiment, the electrode 132 extends downwardly into the interior of the wet sensor cup 114 by at least some predefined distance. Preferably, the electrode 132 is rigid or semi-rigid so that the electrode 132 remains in an extended orientation into the interior of the wet sensor cup 114 during use. When a first portion of the electrically conductive wet gel 122 is squeezed out from the wet sensor cup 114, through the optional sponge 140, and onto the surface of the electrically conductive element 106, a remaining second portion of the electrically conductive wet gel 122 remains within the wet sensor cup 114 to remain in electrical contact with the downwardly extending electrode 132. The first portion of the electrically conductive wet gel remains in electrical contact with the second portion of the electrically conductive wet gel.

In some embodiments, a small amount of the electrically conductive wet gel 122 or another electrically conductive material may reside in the gap 142 prior to electrically coupling the wet sensor lead component 104 to the dry sensor component 102. Alternatively, or additionally, the practitioner may apply a small amount of the electrically conductive wet gel 122 or another electrically conductive material into the gap 142 prior to electrically coupling the wet sensor lead component 104 to the dry sensor component 102.

In the various embodiments, the wet sensor lead component 104 is not designed to contact the skin of the patient, although inadvertent contact is possible and acceptable. In such instances, the electrically conductive wet gel 122 is not harmful to the patient or their skin. Such inadvertent contact with the patient's skin of the wet sensor cup 114, and/or the electrically conductive wet gel 122, does not adversely affect the conductivity of the dry sensor component 102 with the wet sensor lead component 104.

In practice, the dry sensor component 102 is first applied to the patient following a non-abrasive skin preparation, or alternately with no skin preparation. After the dry sensor component 102 has been secured to the patient, the lower surface of the electrically conductive element 106 is in physical and electrical contact with the patient's skin.

Then, the wet sensor cup 114 of the wet sensor lead component 104 is applied directly onto a portion of the electrically conductive element 106 (or one of the dry conductor leads 112). If a perforated or liquid permeable thick film covering 108 covers the electrically conductive element 106 (or one of the dry conductor leads 112), then the wet sensor cup 114 may be applied onto the top surface of the thick film covering 108. After the wet sensor cup 114 is secured to the outside surface of the dry sensor component 102, the viscous electrically conductive wet gel 122 is transported so as to come into electrical and physical contact with the electrically conductive element 106 (or one of the dry conductor leads 112) of the dry sensor component 102.

Following application of the dry sensor component 102 to the patient's skin, followed by placement of the wet sensor lead component 104 onto the dry sensor component 102, and then attachment of the wet sensor lead component 104 to the desired recording or stimulating device (amplifier), the hybrid biosensor 100 is ready for performance or a performance check. Following the optional performance check, the hybrid biosensor 100 is ready for clinical use.

Many advantages are provided by embodiments of the hybrid biosensor 100. The dry sensor component 102, or multiple dry sensor components 102, may be secured to the patient at a first location in a clinic, hospital, doctor's office, testing facility, or the like. Then, the patient may walk to, or be transported to, a second different location where the test procedure is to be performed. For example, the amplifier 110 may at the second location. (Alternatively, the hybrid biosensor 100 may be wirelessly communicatively coupled to the remotely located amplifier 110.) The patient testing may then be performed at the second location. Here, many different patients may have their one or more dry sensor components 102 secured to their body, and then be tested during a shortened test period since the wet sensor lead components 104 can then be quickly and conveniently secured to the dry sensor component(s) 102 on the patient that is being currently tested. That is, a single amplifier 110 may be used to serially test a plurality of different patients who have previously had the dry sensor component 102 secured to their skin. Since the wet sensor lead component 104 is a single use component, the used wet sensor lead component 104 may be discarded. If a plurality of dry conductor leads 112 are used, a new wet sensor lead component 104 may later be secured to an unused dry conductor lead 112 of the dry sensor component 102.

Another advantage is realized when a patient must undergo a variety of different testes using different amplifiers 110. A single dry sensor component 102 having a plurality of dry conductor leads 112 may be secured to multiple different wet sensor lead components 104. The different wet sensor lead components 104 may be secured to different amplifiers 110. Here, the patient is only subjected to a single dry sensor component 102 for the multiple and/or different tests. Such tests may be performed concurrently and/or sequentially. Sequential tests may even be performed at different locations.

When a test (interchangeably referred to herein as a clinical use session) has been completed, the wet sensor lead component 104 can be detached from the dry sensor component 102 (or the dry conductor lead 112, if used) without removing the entirety of the dry sensor component 102 from the patient's skin, even if the gel of the wet sensor lead component 104 locally damages the peninsula 112 or another portion of the wet sensor lead component 104.

One skilled in the art appreciates that maintaining a sterile environment in any health facility where patient testing is of paramount importance. Accordingly, prior to use, the dry sensor component 102 is enclosed within a sterile package. When the patient's skin has been prepared, the dry sensor component 102 may be removed from its sterile packaging. Optionally, a flexible protective and sterile removeable film 124 (see FIG. 3) may be pre-secured to the bottom surface of the dry sensor component 102. Here, the practitioner may peel away and discard the film 124 and immediately secure the sterile bottom surface of the dry sensor component 102 to the patient's skin. Further, prior to application of the dry sensor component 102 to the patient's skin, the film 124 protects the surface of the electrically conductive element 106 from inadvertent contamination and/or physical damage.

All or portions of the upper surface of the dry sensor component 102 may be similarly protected by a flexible protective and sterile removeable film 124. Alternatively, or additionally, in a preferred embodiment, each of the one or more protruding dry conductor leads 112 are individually covered by a flexible protective and sterile removeable film 126 (see FIG. 3). After the dry sensor component 102 has been secured to the patient's skin, the film 126 remains to cover and protect each individual dry conductor lead 112 prior to use. When the practitioner is ready to couple a wet sensor cup 114 of a wet sensor lead component 104 to the dry sensor component 102, the practitioner simply peels away the film 126, and then secures the wet sensor cup 114 to the newly exposed surface of the dry conductor lead 112. A pull tab may be included on the edge of the film 126 (and the other films) for easy grasping by the practitioner.

In a preferred embodiment, a flexible protective and sterile removeable film 128 (see FIG. 3) is pre-secured to the lower surface of the wet sensor cup 114. Here, immediately prior to use, the wet sensor lead component 104 may be removed from its sterile packaging. The practitioner may then peel away and remove the film 128 from the lower surface of the wet sensor cup 114, and then secure the wet sensor cup 114 to the exposed surface of the electrically conductive element 106 or to a selected one of the dry conductor leads 112. Use of the film 128 maintains sterility of the wet sensor cup 114 prior to use. Another benefit of the flexible protective and sterile removeable film 128 on the bottom of the wet sensor cup 114 is that the electrically conductive wet gel 122 is retained within the interior of the wet sensor cup 114 prior to use.

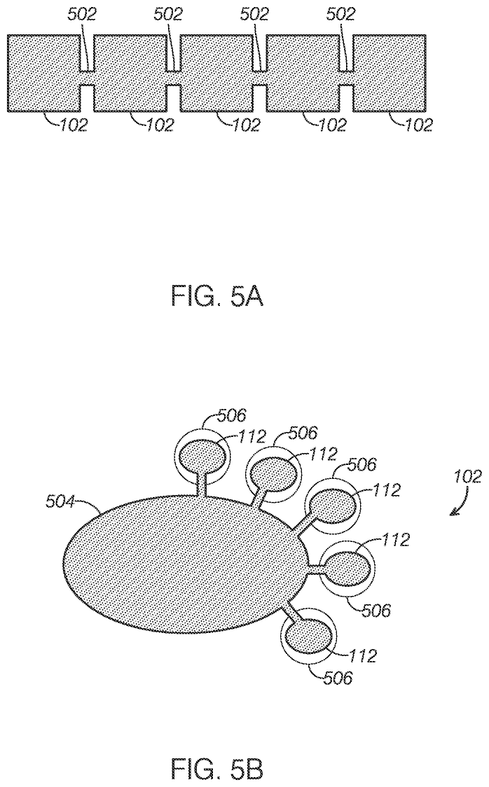

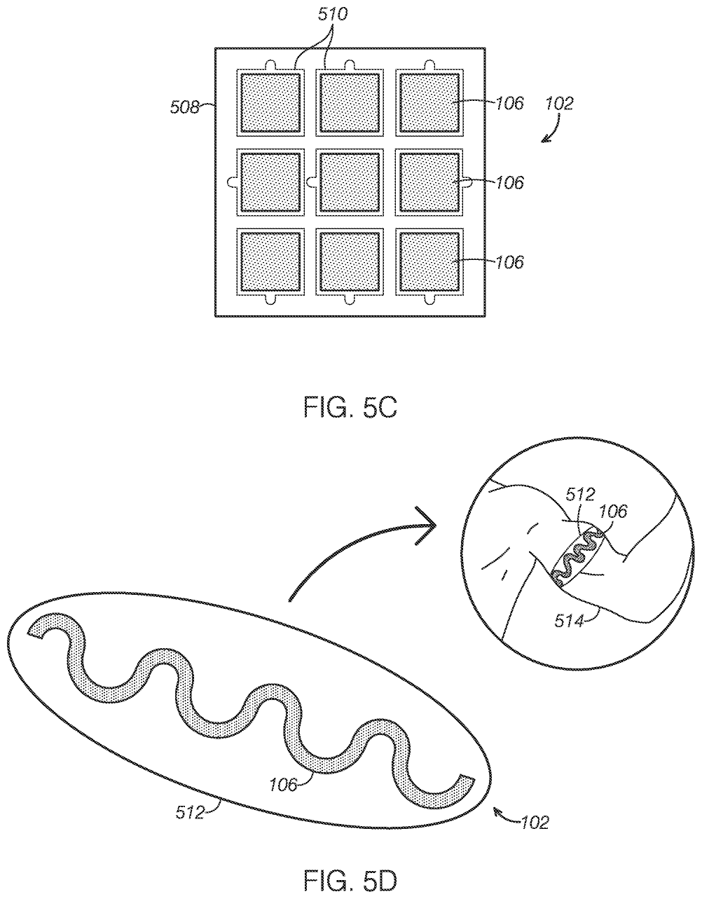

FIGS. 5A-5D are diagrams of various embodiments of the dry sensor component 102. A dry sensor component 102 may be constructed in a variety of shape and sizes. The example embodiment illustrated in FIG. 5A shows a plurality of dry sensor components 102 separated from each other by a breakaway 502. With this embodiment, the plurality of dry sensor components 102 may be provided as a strip or roll. The practitioner may select the number of dry sensor components 102 needed for testing a particular patient, separate the selected dry sensor components 102 from the roll or strip by severing the breakaways 502, and then apply the dry sensor components 102 to the patient at desired locations. Several dry sensor components 102 may also be applied at the same location to allow recording consistently from the same location over many recording sessions. The unused dry sensor components 102 can be retained in their sterile package. In some embodiments, each individual dry sensor component 102 may include one or more dry conductor leads 112 (not shown).

The example embodiment illustrated in FIG. 5B shows a dry sensor component 102 with five dry conductor leads 112 extending outwardly from the body 504 of the electrically conductive element 106. After the dry sensor component 102 is secured to the patient's skin so that the body 504 of the electrically conductive element 106 is in electrical and physical contact with the patient's skin, up to five different wet sensor lead components 104 may be secured to selected ones of the five dry conductor leads 112. The dry conductor leads 112 may or may not be in electrical and/or physical contact with the patient's skin. In some embodiments, a lower protective covering 506 may be disposed on the lower side of each of the dry conductor leads 112 to provide support and to protect the dry conductor leads 112 from inadvertent damage and/or contamination.

In some embodiments, to accommodate local damage, the peninsula 112 of the dry sensor may be constructed with mechanically weak points that function as breakaways. After use, the peninsula 112 that was coupled to a wet sensor lead component 104 may be easily removed, while the remaining portion of the dry sensor component 102 may remain secured to the patient's skin.

The example embodiment illustrated in FIG. 5C shows a dry sensor component 102 with a plurality of electrically conductive material elements 106 on a substrate 508 and separated from each other by a space. In some embodiments, the substrate 508 may be, or may be part of, the thick film covering 108. After securing the dry sensor component 102 to the patient, each of the electrically conductive material elements 106 are in electrical and physical contact with the patient's skin. Preferably, each of the electrically conductive material elements 106 are individually covered with a peel-away protective film 510. Prior to each use, the practitioner peels away the film 510 and then applies a single-use wet sensor lead component 104 to the newly exposed electrically conductive element 106 element. Accordingly, a plurality of different tests may be concurrently and/or sequentially conducted on the patient using the single dry sensor component 102.

The example embodiment illustrated in FIG. 5D is an expandable (stretchable) dry sensor component 102. An elongated curved electrically conductive element 106 element is disposed on a flexible substrate. The expandable dry sensor component 102 is configured to stretch over and/or around a portion of the patient's body. Here, the expandable dry sensor component 102 is designed to increase expansion capabilities over parts of the body that undergo a great deal of skin expansion and/or movement. For example, the expandable dry sensor component 102 is conceptually illustrated as being secured to a patient's arm 514. As the patient moves and/or flexes their arm, the expandable dry sensor component 102 deforms so as to maintain electrical and physical contact with the skin of the patient's arm. This example embodiment allows recording over muscles that experience large shape changes without damaging the dry sensor component 102.

The electrically conductive element 106 of a dry sensor component 102 may be chemically modified to provide molecule-specific detection. If the electrically conductive element 106 is graphene and the graphene has been modified to detect specific molecules, the hybrid biosensor 100 will retain the ability to detect those molecules. Alternatively, or additionally, a dry sensor component 102 may be unmodified to function as a voltage sensor.

The electrically conductive element 106 of a dry sensor component 102 may be made using a thin film containing conductors that include metal, such as gold, or graphene, or compounds composed of a graphene base structure. The electrically conductive element 106 and/or the dry sensor component 102 may be any shape, including circles and polygons, with non-limiting example widths that vary between 0.1 mm and 10 cm or more. The dry sensor component 102 may be without holes or may contain holes that outline open spaces or areas.

One or more of the dry sensor components 102 may remain on the patient for extended periods of time, whether they are used for recording or are currently being used for recording. If removed, some embodiments of the wet sensor lead component 104 may optionally be reapplied to an unused dry sensor component 102 when repeat recording is desired. Alternately, the dry sensor component 102 may be applied prior to intended use, verified for correct functioning (performance check, below), then the wet sensor lead component 104 may be applied later, when a first recording is desired.

An unexpected advantage realized by embodiments of the hybrid biosensor 100 is that a dry sensor component 102 may remain on the patient and function for extended periods, such as hours, days, or weeks. The wet sensor lead component 104 may be detached from the dry sensor component 102 and the function of the dry sensor component 102 remains intact. Sequential data readings and/or recordings may be accomplished by reapplying the wet sensor lead component 104, or a different or new wet sensor lead component 104, to the same dry sensor component 102.

In some cases, the hybrid biosensor 100 may provide for an internal performance check. In these cases, performance of the dry sensor component 102 may be checked. Performance checking is accomplished by briefly touching a wet lead containing a low-adherence gel or electrolyte solution to the dry sensor and measuring the impedance of the dry sensor component 102 using an impedance meter. A second common ground sensor is preferably attached to the patient for the performance check, but may be of any construction, legacy or hybrid. If the measured impedance is below threshold, the dry sensor component 102 is performing normally.

As another example, if a recording session is planned for future time, the dry sensor component 102 may be applied and tested for correct functioning without performing a recording. When recording is desired (repetitively or initially after hours, days, weeks), the wet sensor lead component 104 may be applied to the dry sensor component 102 and a first recording or subsequent recording initiated.

An alternative embodiment of the hybrid biosensor 100 may be configured as a hybrid electrode using a dry sensor component 102 and a wet sensor lead component 104. An electrode embodiment may be used to pass electrical current into the patient. Hybrid electrodes may be constructed of the same materials and in the same manner as passive sensors, but with dimensions capable of passing electrical current from the wet sensor lead component 104 to the dry sensor component 102. Electric current passed into the patient with the hybrid electrode 100 may stimulate muscle activity or decrease sensory nerve functioning and pain sensations. The electrode may be any combination of silver pellet or silver wire that has been prepared with a silver chloride layer, such as, but not limited to Ag/AgCL.

Various clinical uses are envisioned for embodiments of the hybrid biosensor 100. A passive sensor embodiment (primarily used for monitoring EEG, ECG, or EMG) may be used for long-duration monitoring in neonatal ICU, repetitive and long-duration cardiac monitoring (repetitive Holter monitoring), repetitive monitoring of patients at home, or repetitive in-office visits, monitoring activities of specific skeletal muscle groups, including muscles that change size or shape when contracting, or biofeedback. An active sensor embodiment may be used for transcutaneous electrical nerve stimulation (TENS), Functional electrical stimulation (FES), or conditioning skin to allow penetration of therapeutic drugs.

The patient may be a human subject or an animal. Accordingly, when the patient is an animal, a veterinarian may test the animal. Alternatively, embodiments of the hybrid biosensor 100 may be used on inanimate objects.

It should be emphasized that the above-described embodiments of the hybrid biosensor 100 are merely possible examples of implementations of the invention. Many variations and modifications may be made to the above-described embodiments. All such modifications and variations are intended to be included herein within the scope of this disclosure and protected by any later filed claims.

Furthermore, the disclosure above encompasses multiple distinct inventions with independent utility. While each of these inventions has been disclosed in a particular form, the specific embodiments disclosed and illustrated above are not to be considered in a limiting sense as numerous variations are possible. The subject matter of the inventions includes all novel and non-obvious combinations and subcombinations of the various elements, features, functions and/or properties disclosed above and inherent to those skilled in the art pertaining to such inventions. Where the disclosure or subsequently filed claims recite “a” element, “a first” element, or any such equivalent term, the disclosure or claims should be understood to incorporate one or more such elements, neither requiring nor excluding two or more such elements.

Applicant(s) reserves the right to submit claims directed to combinations and subcombinations of the disclosed inventions that are believed to be novel and non-obvious. Inventions embodied in other combinations and subcombinations of features, functions, elements and/or properties may be claimed through amendment of those claims or presentation of new claims in the present application or in a related application. Such amended or new claims, whether they are directed to the same invention or a different invention and whether they are different, broader, narrower, or equal in scope to the original claims, are to be considered within the subject matter of the inventions described herein.

Therefore, having thus described the invention, at least the following is claimed:

1. A hybrid biosensor configured to be secured to a patient's skin to detect voltage information that is communicated to an amplifier, comprising:
a dry sensor component configured to be in electrical contact with the patient's skin after the dry sensor component has been secured to the patient's skin; and
a wet sensor lead component configured to be secured to the dry sensor component after the dry sensor component has been secured to the patient's skin,
wherein the wet sensor lead component, when communicatively coupled to the dry sensor component and to the amplifier, communicates the voltage information sensed by the dry sensor component to the amplifier.

2. The hybrid biosensor of claim 1, wherein the dry sensor component comprises:
an electrically conductive element that is in electrical contact with the patient's skin after the dry sensor component has been secured to the patient's skin.

3. The hybrid biosensor of claim 2, wherein the wet sensor lead component comprises:
a wet sensor cup;
an electrically conductive wet gel residing within an interior of the wet sensor cup; and
an electrode residing within the wet sensor cup.

4. The hybrid biosensor of claim 3,
wherein the electrically conductive wet gel is electrically connected to the electrode, and
wherein the electrically conductive wet gel is electrically connected to the electrically conductive element after the wet sensor lead component is secured to the dry sensor component.

5. The hybrid biosensor of claim 4,
wherein the electrode extends into the interior of the wet sensor cup,
wherein a first portion of the electrically conductive wet gel is transported to the electrically conductive element in response to securing the wet sensor lead component to the dry sensor component after the dry sensor component has been secured to the patient's skin,
wherein a second portion of the electrically conductive wet gel remains electrically connected to the electrode extended into the interior of the wet sensor cup after the first portion of the electrically conductive wet gel is transported to the electrically conductive element of the dry sensor component, and
wherein the first portion of the electrically conductive wet gel remains in electrical contact with the second portion of the electrically conductive wet gel.

6. The hybrid biosensor of claim 4, wherein the wet sensor cup further comprises:
a sponge residing proximate to a bottom of the wet sensor cup,
wherein a first portion of the electrically conductive wet gel is transported through the sponge to the electrically conductive element in response to securing the wet sensor lead component to the dry sensor component after the dry sensor component has been secured to the patient's skin,
wherein a second portion of the electrically conductive wet gel remains electrically connected to the electrode after the first portion of the electrically conductive wet gel is transported to the electrically conductive element of the dry sensor component, and
wherein the first portion of the electrically conductive wet gel remains in electrical contact with the second portion of the electrically conductive wet gel.

7. The hybrid biosensor of claim 6,
wherein the sponge is defined by an outer perimeter edge that corresponds to an inside of a perimeter of the bottom of the wet sensor cup,
wherein during assembly of the wet sensor lead component, the electrically conductive wet gel is inserted into the interior of the wet sensor cup, and
wherein the sponge is inserted into the bottom of the wet sensor cup after the electrically conductive wet gel is inserted into the interior of the wet sensor cup.

8. The hybrid biosensor of claim 7, wherein the wet sensor cup comprises:
a cup wall that defines the interior of the wet sensor cup and the perimeter of the bottom of the wet sensor cup,
wherein the cup wall is made of a deformable material, and
wherein the first portion of the electrically conductive wet gel is urged through the sponge to become in electrical contact with the electrically conductive element of the dry sensor component when the cup wall is deformed in response to securing the wet sensor lead component to the dry sensor component.

9. The hybrid biosensor of claim 7, wherein the wet sensor cup comprises:
a film detachably secured to the bottom of the wet sensor cup,
wherein the detachably secured film retains the first portion of the electrically conductive wet gel within the interior of the wet sensor cup until the film is detached from the bottom of the wet sensor cup.

10. The hybrid biosensor of claim 3, wherein the wet sensor cup comprises:
a connector secured to an outside surface of the wet sensor cup; and
a wet sensor lead wire with a proximal end electrically connected to the connector,
wherein a distal end of the wet sensor lead wire is configured to electrically connect to a wire connector that extends back to the amplifier.

11. The hybrid biosensor of claim 3, wherein the dry sensor component comprises:
a film of electrically conductive material.

12. The hybrid biosensor of claim 3, wherein the dry sensor component comprises:
a thick film covering the electrically conductive element,
wherein the thick film covering provides support and protection to the electrically conductive element.

13. The hybrid biosensor of claim 12:
wherein the thick film is electrically insulative,
wherein a portion of the electrically conductive wet gel that has passed into the thick film is electrically coupled to the electrically conductive element, and
wherein the portion of the electrically conductive wet gel that has passed into the thick film covering becomes electrically connected to the electrically conductive wet gel in response to securing the wet sensor lead component to the dry sensor component.

14. The hybrid biosensor of claim 12:
wherein the thick film covering is an electrical insulator so that the thick film is not electrically coupled to the electrically conductive element, and
wherein a portion of the thick film covering is removeable to expose the electrically conductive element so that the electrically conductive element becomes electrically connected to the electrically conductive wet gel in response to securing the wet sensor lead component to the dry sensor component.

15. The hybrid biosensor of claim 12:
wherein the thick film covering is porous, and
wherein a portion of the electrically conductive wet gel is transported through the porous thick film covering so that the electrically conductive element becomes electrically connected to the electrically conductive wet gel in response to securing the wet sensor lead component to the dry sensor component.

16. The hybrid biosensor of claim 12:
wherein the thick film covering is stretchable, and
wherein electrically conductive element remains electrically connected to the electrically conductive wet gel when the thick film covering is stretched.

17. The hybrid biosensor of claim 3, wherein the electrically conductive element comprises:
a dry conductor lead extending outwardly from the electrically conductive element,
wherein the electrically conductive element becomes electrically connected to the electrically conductive wet gel in response to securing the wet sensor cup to the dry conductor lead.

18. The hybrid biosensor of claim 3, wherein the wet sensor cup is a first wet sensor cup of a plurality of wet sensor cups of a plurality of wet sensor lead components, and wherein the electrically conductive element comprises:
a first dry conductor lead extending outwardly from the electrically conductive element; and
a second dry conductor lead extending outwardly from the electrically conductive element,
wherein the electrically conductive element becomes electrically connected to the electrically conductive wet gel of the first wet sensor cup in response to securing the first wet sensor cup to the first dry conductor lead, and
wherein the electrically conductive element becomes electrically connected to the electrically conductive wet gel of a second wet sensor cup in response to securing the second wet sensor cup to the second dry conductor lead.

19. The hybrid biosensor of claim 3,
wherein the dry sensor component is a first one of a plurality of dry sensor components, and
wherein the first dry sensor component is coupled to a second dry sensor component by a breakaway.

20. The hybrid biosensor of claim 3,
wherein the electrically conductive element is a first one of a plurality of electrically conductive elements,
wherein the wet sensor cup is a first wet sensor cup of a plurality of wet sensor cups of a plurality of wet sensor lead components,
wherein the first electrically conductive element becomes electrically connected to the electrically conductive wet gel of the first wet sensor cup in response to securing the first wet sensor cup to the first electrically conductive element, and
wherein the second electrically conductive element becomes electrically connected to the electrically conductive wet gel of a second wet sensor cup in response to securing the second wet sensor cup to the second electrically conductive element.

21. A method of using a hybrid biosensor configured to be secured to a patient's skin to detect voltage information that is communicated to an amplifier, comprising:
securing a dry sensor component to a patient's skin, wherein the dry sensor component is configured to be in electrical contact with the patient's skin after the dry sensor component has been secured to the patient's skin; and
securing a wet sensor lead component to the dry sensor component after the dry sensor component has been secured to the patient's skin,
wherein the wet sensor lead component, when communicatively coupled to the dry sensor component and to the amplifier, communicates the voltage information sensed by the dry sensor component to the amplifier.
